(19) United States
(12) Patent Application Publication
(10) Pub. No.: US 2010/0129339 A1
(43) Pub. Date: May 27, 2010

(54) NKT CELL-STIMULATING AGENT FOR ADMINISTRATION THROUGH UPPER RESPIRATORY TRACT MUCOUS MEMBRANE

(75) Inventors: Masaru Taniguchi, Yokohama-shi (JP); Shigetoshi Horiguchi, Chiba-shi (JP); Yoshitaka Okamoto, Chiba-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: RIKEN, Wako-shi (JP); NATIONAL UNIVERSITY CORPORATION CHIBA UNIVERSITY, Chiba-shi (JP)

(21) Appl. No.: 12/089,230

(22) PCT Filed: Oct. 6, 2006
(86) PCT No.: PCT/JP2006/320424

§ 371 Date: Jun. 26, 2008

(30) Foreign Application Priority Data
Oct. 6, 2005 (JP) 2005-294124

(51) Int. Cl. A61K 45/00; A61P 37/04

(52) U.S. Cl. 424/93.71

(57) ABSTRACT

The present invention provides an NKT cell stimulating agent containing antigen-presenting cells pulsed with an NKT cell ligand, to be administered submucosally in the upper airway. By submucosal administration in the upper airway, it is possible to stimulate NKT cells and stimulate immune reactions extremely efficiently with a small number of NKT cell ligand-pulsed antigen-presenting cells. By submucosal administration in the upper airway, NKT cells can be induced selectively in cervical lymph nodes.

### TECHNICAL FIELD

The present invention relates to an NKT cell stimulating agent to be administered submucosally in the upper airway and the like. More specifically, the present invention relates to an NKT cell stimulant (or an inducer of NKT cells in cervical lymph nodes, inducer of interferon γ production, immunostimulant and the like) containing antigen-presenting cells pulsed with an NKT cell ligand, to be administered submucosally in the upper airway and the like.

### BACKGROUND ART

For advanced cases in stage III and stage IV of head and neck squamous cell carcinomas, as a rule, triple combination therapy comprising surgery, radiation, and chemotherapy is performed. With respect to surgical treatment, particularly after the last half of the 1980s, autologous tissue transplantation of free flaps, intestine, and bone with vascular stalk spread, extended resection became a relatively easy procedure, some effects were obtained for functional and morphological retention, and local control improved remarkably [Yoshitaka Okamoto, Treatment of advanced head and neck cancer—Measures and problems, Jibi-Rinsho 94:577-585, 2001]. Even absolute resection of cancers infiltrating the internal carotid artery or skull base became possible [Yoshitaka Okamoto, Challenging cancers infiltrating the internal carotid artery, Jibiten 42:232:239, 1999, Chazono H, Okamoto Y, Matsuzaki Z, Ogino J, Endo S, Matsuoka T, Horikoshi T, Nukui H, Hadeishi H, Yasui N, Extra-intracranial bypass for reconstruction of internal carotid artery in the management of head and neck cancer. Ann Vasc Surg 17: 260-265, 2003]. However, extending the range of resection leads to limitations on functional and morphological retention by reconstructive surgery, and causes remarkable deterioration of the QOL of patients. In stage IV, a combination of radiation and chemotherapy is indispensable to improve therapeutic outcomes; however, in stage IV, for N2c and N3 cases and cases of infiltration in the carotid artery, therapeutic outcomes were poor even with extended resection, the 5-year survival rate being lower than 50% [Okamoto Y, Inugami A, Matsuzaki Z, Yokomizo M, Konno A, Togawa K, Kuribayashi K, Ogawa T, Kanno I, Carotid artery resection for head and neck cancer. Surgery 120: 54-59, 1996]. In a treatment comprising extended resection followed by a combination of radiation and chemotherapy, the survival rate improved significantly, but functional preservation for the larynx and the like was difficult.

Meanwhile, in Japan, since the launch of a platinum preparation in 1985, with the expectation for high efficacy of chemotherapy, the preparation has been used as a neo-adjuvant or adjuvant therapy [Okamoto Y, Konno A, Togawa K, Kato T, Tamakawa Y, Amano Y, Arterial chemoembolization with cisplatin microcapsules. Br J Cancer 53: 369-375, 1986, Tomura N, Kobayashi M, Watarai J, Okamoto Y, Togawa K, Chemoembolization of head and neck cancer with carboplatin-microcapsules. Acta Radiologica 37: 52-56, 1996]. However, as a result of randomized studies in Europe and the US, by about 10 years previously, it had been nearly concluded that neo-adjuvant treatment does not contribute to the improvement in survival rate, compared with radiation monotherapy, though it is somewhat effective for functional preservation [Rischin D, Head and neck cancer debate: Does induction chemotherapy remain a worthy approach? Am Soc Clin Oncol: 300-304, 2003]. Currently, concurrent radiation and chemotherapy is attracting attention as the central treatment for triple combination therapy, and randomized studies have reported that this therapy is more likely to achieve functional retention than radiation monotherapy, and also contributes to an improvement in survival rate [Adelstein D J, Layertu P, Saxton J P, Secic M, Wood B G, Wanamaker J R, Eliachar I, Strome M, Larto M A, Mature results of a phase III randomized trial comparing concurrent chemotherapy with radiation alone in patients with stage III and IV squamous cell carcinoma of the head and neck cancer 88: 876-883, 2000]. However, the improvement in survival rate is up to 0 to 8%, the 5 years survival rate being about 20 to 40%; moreover, many studies have excluded N2c, N3, or advanced T4 cases from the study populations. Additionally, the results of salvage surgery are poor [Forastiere A A, Goepfert H, Maor M, Pajak T, Weher R, Morrison M, Glisson B, Trotti A, Ridge J A, Chao C, Peters G, Lee D J, Leaf A, Ensky J, Cooper J, Concurrent Chemotherapy and radiotherapy for organ preservation in advanced laryngeal cancer. N Engl J Med 349: 2091-2098, 2003].

Hence, the treatment of advanced head and neck squamous cell carcinomas, whether by surgery, radiation, or chemotherapy, poses major problems. To improve the results, and to lessen the burden on patients, a new therapeutic strategy is indispensable [Yoshitaka Okamoto, Treatment of head and neck cancer: Problems and management, Chiba-Igaku 79:1-5, 2003]. Although conventional cellular immunotherapy is highly safe, only a very limited effect has been obtained.

NKT cells are unique cells expressing both a T cell receptor (TCR) and an NK cell receptor (NKR) on the same cell surface, and were for the first time reported as the fourth lymphocytes distinct from T cells, B cells, and NK cells [Fowlkes B J, Kruisbeek A M, Ton-That H, Weston M A, Coligan J E, Schwartz R H, Pardoll D M, A novel population of T-cell receptor alpha beta-bearing thymocytes which predominantly expresses a single V beta gene family. Nature 1987 Sep. 17-23; 329(6136): 251-4, Budd R C, Miescher G C, Howe R C, Lees R K, Bron C, MacDonald H R, Developmentally regulated expression of T cell receptor beta chain variable domains in immature thymocytes. J Exp Med 1987 Aug. 1; 166(2): 577-82, Imai K, Kanno M, Kimoto H, Shigemoto K, Yamamoto S, Taniguchi M, Sequence and expression of transcripts of the T-cell antigen receptor alpha-chain gene in a functional, antigen-specific suppressor-T-cell hybridoma. Proc Natl Acad Sci USA 1986 November; 83(22): 8708-12]. The T cell antigen receptor (TCR) on NKT cells is composed of an extremely limited α chains (Vα14-Jα281 in mice, Vα24-JαQ in humans) and β chains (Vβ8, Vβ7 or Vβ2 in mice, Vβ11 in humans) [Dellabona P, Padovan E, Casorati G, Brockhaus M, Lanzavecchia A, An invariant V alpha 24-J alpha Q/V beta 11 T cell receptor is expressed in all individuals by clonally expanded CD4⁻8⁻ T cells, J Exp Med 1994 Sep. 1; 180(3):1171-6, Porcelli S, Gerdes D, Fertig A M, Balk S P, Human T cells expressing an invariant V alpha 24-J alpha Q TCR alpha are CD4⁻ and heterogeneous with respect to TCR beta expression, Hum Immunol 1996 June-July; 48(1-2): 63-7, Makino Y, Kanno R, Ito T, Higashino K, Taniguchi M, Predominant expression of invariant V alpha 14⁺ TCR alpha chain in NK1.1⁺ T cell populations. Int Immunol 1995 July; 7(7): 1157-61, Taniguchi M, Koseki H, Tokuhisa T, Masuda K, Sato H, Kondo E, Kawano T, Cui J, Perkes A, Koyasu S, Makino Y, Essential requirement of an invariant V alpha 14 T cell antigen receptor expression in the development of natural killer T cells. Proc Natl Acad Sci USA 1996 Oct. 1; 93(20): 11025-8, Makino Y, Kanno R, Koseki H, Taniguchi M, Development of Valpha14⁺ NK T cells in the early stages of embryogenesis. Proc Natl Acad Sci USA 1996 Jun. 25; 93(13): 6516-20], and it has been demonstrated that the molecule recognized thereby is the CD1d molecule, which is an antigen-presenting molecule similar to MHC class I [Bendelac A, Lantz O, Quimby M E, Yewdell J W, Bennink J R, Brutkiewicz R R, CD1 recognition by mouse NK1+ T lymphocytes, Science 1995 May 12; 268(5212): 863-5, Adachi Y, Koseki H, Zijlstra M, Taniguchi M, Positive selection of invariant V alpha 14⁺ T cells by non-major histocompatibility complex-encoded class I-like molecules expressed on bone marrow-derived cells. Proc Natl Acad Sci USA 1995 Feb. 14; 92(4): 1200-4]. Recently, it was shown that presentation of α-galactosylceramide, a glycolipid, on CD1d could specifically activate NKT cells [Kawano T, Cui J, Koezuka Y, Toura I, Kaneko Y, Motoki K, Ueno H, Nakagawa R, Sato H, Kondo E, Koseki H, Taniguchi M, CD1d-restricted and TCR-mediated activation of Valpha14 NKT cells by glycosylceramides, Science 1997 Nov. 28; 278(5343): 1626-9, Cui J, Shin T, Kawano T, Sato H, Kondo E, Toura I, Kaneko Y, Koseki H, Kanno M, Taniguchi M, Requirement for Valpha14 NKT cells in IL-12-mediated rejection of tumors. Science 1997 Nov. 28; 278(5343): 1623-6]. NKT cells activated by the ligand promptly produce large amounts of IFNγ and IL-4, and exhibit potent cytotoxic activity via perforin/granzyme B. More recently, it was demonstrated that NKT cells had a unique action mechanism for causing a variety of immune reactions, and as a result, exhibiting a potent antitumor action. It has been reported that α-galactosylceramide exhibited a remarkable antitumor effect dependently on NKT cells in various mouse liver metastasis models [Morita M, Motoki K, Akimoto K, Natori T, Sakai T, Sawa E, YamajiK, Koezuka Y, Kobayashi E, Fukushima H, Structure-activity relationship of alpha-galactosylceramides against B16-bearing mice. J Med Chem 1995 Jun. 9; 38(12): 2176-87, Nakagawa R, Motoki K, Ueno H, Iijima R, Nakamura H, Kobayashi E, Shimosaka A, Koezuka Y, Treatment of hepatic metastasis of the colon26 adenocarcinoma with an alpha-galactosylceramide, KRN7000. Cancer Res 1998 Mar. 15; 58(6): 1202-7, Kawano T, Cui J, Koezuka Y, Toura I, Kaneko Y, Sato H, Kondo E, Harada M, Koseki H, Nakayama T, Tanaka Y, Taniguchi M, Natural killer-like nonspecific tumor cell lysis mediated by specific ligand-activated Valpha14 NKT cells. Proc Natl Acad Sci USA 1998 May 12; 95(10): 5690-3]. It was also found that α-galactosylceramide is capable of specifically activating not only mouse NKT cells, but also human NKT cells [Kawano T, Nakayama T, Kamada N, Kaneko Y, Harada M, Ogura N, Akutsu Y, Motohashi S, Iizasa T, Endo H, Fujisawa T, Shinkai H, Taniguchi M, Antitumor cytotoxicity mediated by ligand-activated human V alpha24 NKT cells. Cancer Res 1999 Oct. 15; 59(20): 5102-5]. On the basis of these results, a phase 1 clinical trial by IV administration of α-galactosylceramide in patients with solid cancers is ongoing in the Netherlands [Giaccone G, Punt C J, Ando Y, Ruijter R, Nishi N, Peters M, von Blomberg B M, Scheper R J, van der Vliet H J, van den Eertwegh A J, Roelvink M, Beijnen J, Zwierzina H, Pinedo H M, A phase I study of the natural killer T-cell ligand alpha-galactosylceramide (KRN7000) in patients with solid tumors. Clin Cancer Res. 2002 Dec. 8: (12); 3702-9].

Dendritic cells (DC) are the most potent antigen-presenting cells in T cell-dependent immune responses. In cancer patients, it is said that by IL-10, VEGF (Vascular Endothelial Growth Factor) and the like secreted from tumors, the maturation, activation and recruitment of DC are inhibited. However, if taken out from the body, induced to the maturation process, pulsed with a tumor-specific antigen to confer a tumor-antigen-specific immune potential, and then infused back to the cancer patient, DC precursor cells are expected to overcome the above-described suppression of the maturation and activation of DC in the body, and to become therapeutically effective. Clinical studies of cancer vaccine therapy with DC (DC therapy) for malignant lymphoma, malignant melanoma, multiple myeloma, prostatic cancer, renal cell carcinoma and the like have already been commenced, and preliminary reports have been presented that induction of antigen-specific cytotoxic T cells (CTL) and tumor shrinkage effect were observed. Reported adverse reactions associated with DC therapy include chills, fever and the like. Worldwide, the development of autoantibodies (anti-thyroidal antibody and the like) and the onset of rheumatoid arthritis have been reported as adverse reactions, but no other serious adverse reactions or complications have been reported; DC therapy is thought to be a relatively safe therapeutic method. However, general DC therapy, which utilizes tumor-specific molecules, poses problems, including efficacy expectable only on a limited kinds of tumors because of the specificity, and the inability to serve as a target for CTL in patients with different MHCs and in cases of reduced expression of MHC class I molecules in tumor cells of the patient, because of MHC restriction.

Meanwhile, on the basis of the above-mentioned antitumor action mechanism of α-galactosylceramide, it was anticipated that an antitumor effect would be obtained by transferring α-galactosylceramide-pulsed DC into a cancer-bearing mouse. From the results of an investigation using animals, it was shown that when the timing of administration of α-galactosylceramide was delayed in a malignant tumor metastasis model, the metastasis suppressing effect disappeared, and that when dendritic cells (DC) pulsed with α-galactosylceramide were administered to a cancer-bearing mouse, lung or liver metastasis was suppressed nearly completely, even when the timing of administration was delayed to some extent [Toura I, Kawano T, Akutsu Y, Nakayama T, Ochiai T, Taniguchi M, Cutting edge: Inhibition of experimental tumor metastasis by dendritic cells pulsed with alpha-galactosylceramide. J Immunol 1999 Sep. 1; 163(5): 2387-91]. This suggests that efficient activation of NKT cells in vivo may be better achieved when α-galactosylceramide is administered in a form being presented on DC, rather than when administered alone.

Furthermore, because the CD1d molecule—NKT cell antigen receptor system, utilized in this therapeutic method, is common to all persons, anyone's NKT cells can be activated with α-galactosylceramide. Additionally, because activated NKT cells exhibit cytotoxic activity irrespective of the expression of MHC class I molecules, this therapy is thought to have an advantage of overcoming a drawback of DC therapy, which comprises pulsing with a cancer-specific peptide.

A safety study for “Therapeutic Method Using α-Galactosyl Cermide (KRN7000)-pulsed Cells in Patients with Recurrent Lung Cancer and Patients with Advanced Lung Cancer” approved by the Chiba University Ethical Committee demonstrated that α-galactosylceramide-pulsed dendritic cell therapy can be performed safely. Also, a safety study for “A Clinical Study Using Activated NKT Cells in Patients with Recurrent Lung Cancer and Patients with Advanced Lung Cancer” demonstrated that intravenous administration of activated NKT cells can be performed safely.

To date, mainly in recurrent cases of lung cancer, intravenous administration of α-galactosylceramide-pulsed dendritic cells has been investigated. In a phase 1 study, experiments were performed with escalation of the number of transferred cells from 5×10⁷/m² for level 1 to 2.5×10⁸/m² for level 2 and 1×10⁹/m² for level 3. As a result, an increased number of peripheral blood NKT cells was observed in one patient receiving level 3 cells out of the 11 patients who participated in the study [Ishikawa A, Motohashi S, Ishikawa E, Fuchida H, Higashino K, Otsuji M, Iizasa T, Nakayama T, Taniguchi M, Fujisawa T, A phase I study of alpha-galactosylceramide (KRN7000)-pulsed dendritic cells in patients with advanced and recurrent non-small cell lung cancer. Clin Cancer Res. 2005 Mar. 1; 11(5): 1910-7]. However, with α-galactosylceramide-pulsed dendritic cells of level 1 or level 2 numbers, no immune responses such as increases in the number of NKT cells in peripheral blood were obtained.

As described above, it is possible to stimulate NKT cells, stimulate immunity, and treat diseases such as tumors more efficiently by administering antigen-presenting cells pulsed with an NKT cell ligand such as α-galactosylceramide, than by administering an NKT cell ligand directly into the body; however, a considerable number of antigen-presenting cells must be used to achieve this effect, which in turn leads to the consumption of large amounts of reagents to prepare the same, posing a problem of increased costs. Additionally, (a) because a large amount of mononuclear cells must be collected from the patient to prepare antigen-presenting cells, (b) because a long time is taken for administration by intravenous drip infusion of a large amount of antigen-presenting cells, and for other reasons, the physical burden on the patient is significant. Amid this situation, there has been a demand for the development of a method of administering antigen-presenting cells that is likely to achieve excellent effects such as NKT cell stimulating action, immunostimulating action, and antitumor action, while reducing the number of antigen-presenting cells used.

Meanwhile, the present inventors reported that when antigen-pulsed dendritic cells were administered submucosally in the nasal cavity, the dendritic cells migrated highly selectively to cervical lymph nodes. The present inventors also confirmed that no NKT cells were detected in normal non-metastatic cervical lymph nodes, whereas a large number of NKT cells were detected in cervical lymph nodes with metastatic head and neck cancer (Shigetoshi Horiguchi, Yoshitaka Okamoto et al., “Migration of Nasal Cavity Mucosal Dendritic Cells in the Body”, Journal of Japan Society of Immunology & Allergology in Otolaryngology, vol. 21, No. 2, p. 10-11, 2003, Chiba University COE Report, Introduction of Cellular Immunotherapy and Heavy-Particle Treatment for Pharyngeal Cancer, p. 116-118, 2005). Therefore, there is a demand for the development of a method of positively inducing NKT cells in lymph nodes before head and neck cancer metastasizes to cervical lymph nodes, and activating antitumor immunity via NKT cells in the cervical lymph nodes.

In view of the above-described circumstances, the present invention is directed to providing a method of administering antigen-presenting cells that makes it possible to stimulate NKT cells, stimulate immunity, and treat diseases such as cancer efficiently and potently using as small a number of antigen-presenting cells as possible in NKT cell ligand-pulsed antigen-presenting cell therapy. The present invention is also directed to providing a method of inducing NKT cells selectively in cervical lymph nodes to activate antitumor immunity via NKT cells in the cervical lymph nodes.

### DISCLOSURE OF THE INVENTION

As a result of extensive investigations to accomplish the above-described objects, the present inventors found that by administering NKT cell ligand-pulsed antigen-presenting cells submucosally in the upper airway, NKT cells, which are usually absent in cervical lymph nodes, are induced selectively in cervical lymph nodes. Furthermore, the present inventors found that by using the method of administration, it is impossible to stimulate NKT cells efficiently with a very small amount of antigen-presenting cells, even in tissues other than cervical lymph nodes (peripheral blood and the like), and to stimulate systemic immune responses, and developed the present invention.

Accordingly, the present invention relates to the following:

(1) An NKT cell stimulating agent containing antigen-presenting cells pulsed with an NKT cell ligand, to be administered submucosally in an upper airway.
(2) The agent according to (1) above, wherein the NKT cell ligand is α-galactosylceramide.
(3) The agent according to (1) above, wherein the upper airway mucosa is a nasal cavity mucosa.
(4) An inducing agent of NKT cells in cervical lymph nodes containing antigen-presenting cells pulsed with an NKT cell ligand, to be administered submucosally in an upper airway.
(5) The agent according to (4) above, wherein the NKT cell ligand is α-galactosylceramide.
(6) The agent according to (4) above, wherein the upper airway mucosa is a nasal cavity mucosa.
(7) An inducing agent of interferon γ production containing antigen-presenting cells pulsed with an NKT cell ligand, to be administered submucosally in an upper airway.
(8) The agent according to (7) above, wherein the NKT cell ligand is α-galactosylceramide.
(9) The agent according to (7) above, wherein the upper airway mucosa is a nasal cavity mucosa.
(10) An immunostimulating agent containing antigen-presenting cells pulsed with an NKT cell ligand, to be administered submucosally in an upper airway.
(11) The agent according to (10) above, wherein the NKT cell ligand is α-galactosylceramide.
(12) The agent according to (10) above, wherein the upper airway mucosa is a nasal cavity mucosa.
(13) A method of stimulating NKT cells, comprising administering antigen-presenting cells pulsed with an NKT cell ligand submucosally in an upper airway.
(14) A method of inducing NKT cells in cervical lymph nodes, comprising administering antigen-presenting cells pulsed with an NKT cell ligand submucosally in an upper airway.
(15) A method of inducing interferon γ production, comprising administering antigen-presenting cells pulsed with an NKT cell ligand submucosally in an upper airway.
(16) A method of stimulating immune reactions, comprising administering antigen-presenting cells pulsed with an NKT cell ligand submucosally in an upper airway.
(17) A use of antigen-presenting cells pulsed with an NKT cell ligand, for producing an NKT cell stimulating agent to be administered submucosally in an upper airway.
(18) A use of antigen-presenting cells pulsed with an NKT cell ligand, for producing an inducing agent of NKT cells in cervical lymph nodes to be administered submucosally in an upper airway.
(19) A use of antigen-presenting cells pulsed with an NKT cell ligand, for producing an inducing agent of interferon γ production to be administered submucosally in an upper airway.
(20) A use of antigen-presenting cells pulsed with an NKT cell ligand, for producing an immunostimulant to be administered submucosally in an upper airway.

With the use of the agent of the present invention, it is possible to stimulate NKT cells, stimulate immune reactions, and treat diseases such as cancer extremely efficiently with a small number of NKT cell ligand-pulsed antigen-presenting cells. This allows a significant reduction in the consumption of reagents used to prepare antigen-presenting cells, thus cutting the costs of the treatment as a whole. Additionally, because the amount of mononuclear cells collected from the patient to prepare antigen-presenting cells can be significantly reduced, and also because the time taken to administer antigen-presenting cells is shortened, the burden on the patient is lessened. Furthermore, because the amount of NKT cell ligand required for the treatment also decreases significantly, safety in the treatment improves further.

Furthermore, with the use of the agent of the present invention, it is possible to induce NKT cells selectively in cervical lymph nodes and activate antitumor immunity via NKT cells in the cervical lymph nodes.

### BRIEF DESCRIPTION OF THE DRAWINGS

FIG. 1 shows the expression of HLA-DR, CD11c and CD86 on the surface of the dendritic cells administered. Numerical figures in gates indicate ratios of positive cells (%).

FIG. 2 shows NKT cells (CD3⁺Vα24⁺Vβ11⁺ cells) (upper panel) and NK cells (CD3⁻CD56⁺ cells) (lower panel) in peripheral blood. Numerical figures in gates indicate ratios of cell counts in each gate (%). The arrow indicates administration of α-GalCer-pulsed dendritic cells.

FIG. 3 shows changes in the numbers of NKT cells and NK cells per ml of peripheral blood.

FIG. 4 shows changes in the number of cells that produced γ interferon in response to α-GalCer stimulation, contained in a peripheral blood mononuclear cell fraction obtained by ELISPOT.

FIG. 5 shows the expression of HLA-DR, CD11c and CD86 on the surface of the dendritic cells administered. Numerical figures in gates indicate ratios of positive cells (%).

FIG. 6 shows NKT cells (CD3⁺Vα24⁺Vβ11⁺ cells) (upper panel) and NK cells (CD3⁻CD56⁺ cells) (lower panel) in peripheral blood. Numerical figures in gates indicate ratios of cell counts in each gate (%). The arrow indicates administration of α-GalCer-pulsed dendritic cells.

FIG. 7 shows changes in the numbers of NKT cells and NK cells per ml of peripheral blood.

FIG. 8 shows changes in the number of cells that produced γ interferon in response to α-GalCer stimulation, contained in a peripheral blood mononuclear cell fraction obtained by ELISPOT.

FIG. 9 shows the induction of NKT cells in cervical lymph nodes by submucosal administration of α-GalCer-pulsed dendritic cells in nasal cavity.

FIG. 10 shows the results of detection of NKT cells in peripheral blood and lymph nodes.

### BEST MODE FOR CARRYING OUT THE INVENTION

The present invention provides an agent (NKT cell stimulating agent, inducing agent of NKT cells in cervical lymph nodes, inducing agent of interferon γ production or immunostimulating agent) containing antigen-presenting cells pulsed with an NKT cell ligand, to be administered submucosally in the upper airway. By submucosal administration in the upper airway, it is possible to stimulate NKT cells, induce interferon γ production, and stimulate immune reactions extremely efficiently with a small number of NKT cell ligand-pulsed antigen-presenting cells. By administering antigen-presenting cells pulsed with an NKT cell ligand submucosally in the upper airway, it is possible to induce NKT cells selectively in cervical lymph nodes.

NKT cells are a kind of lymphocytes expressing two antigen receptors, i.e., T cell receptor (TCR) and NK receptor. NKT cells recognize the following “NKT cell ligand” presented on CD1 (for example, CD1d) molecules via the T cell receptor on the NKT cells. The repertoire of T cell receptors on NKT cells, unlike on ordinary T cells, are extremely limited. For example, the α chain of the T cell receptor on mouse NKT cells (sometimes referred to as Vα14NKT cells) is encoded by invariant Vα14 and Jα281 gene segments (Proc Natl Acad Sci USA, 83, p. 8708-8712, 1986; Proc Natl Acad Sci USA, 88, p. 7518-7522, 1991; J Exp Med, 180, p. 1097-1106, 1994), not less than 90% of the β chain is Vβ8, and a limited repertoire of Vβ7 and Vβ2 can be contained. The T cell receptor on human NKT cells is known to be a combination of invariant Vα24, which is highly homologous to mouse Vα14, and Vβ11, which is closely related to Vβ8.2.

“An NKT cell ligand” refers to a compound capable of being recognized specifically by a T cell receptor on NKT cells and specifically activating NKT cells when presented onto a CD1 molecule. Examples of “NKT cell ligands” used in the present invention include α-glycosylceramide, isoglobotrihexosylceramide (Science, 306, p. 1786-1789, 2004), OCH (Nature 413:531, 2001) and the like. α-glycosylceramide is a sphingoglycolipid comprising a saccharide, such as galactose or glucose, and a ceramide, bound in a configuration, and it is exemplified by those disclosed in WO 93/05055, WO94/02168, WO94/09020, WO94/24142 and WO98/44928, Science, 278, p. 1626-1629, 1997 and the like can be mentioned. In particular, (2S,3S,4R)-1-O-(α-D-galactopyranosyl)-2-hexacosanoylamino-1,3,4-octadecanetriol (herein referred to as α-galactosylceramide or α-GalCer) is preferable.

Herein, the term “NKT cell ligand” is used with a meaning including salts thereof. Useful salts of NKT cell ligands include salts with physiologically acceptable acids (e.g., inorganic acids, organic acids), or bases (e.g., alkali metal salts) and the like, and physiologically acceptable acid addition salts are particularly preferable. Examples of such salts include salts with inorganic acids (for example, hydrochloric acid, phosphoric acid, hydrobromic acid, sulfuric acid), or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

Herein, the term “NKT cell ligand” is used with a meaning including solvates thereof (hydrates and the like).

An antigen-presenting cell refers to a cell that presents an antigen to lymphocytes to promote the activation of the lymphocytes. Usually, antigen-presenting cells are dendritic cells or macrophages capable of presenting an antigen to T cells or NKT cells. Particularly, dendritic cells have the potent capability of antigen presentation, and are capable of presenting an antigen via MHC Class I, MHC Class I-like molecules (CD1 and the like), MHC Class II and the like expressed on the cell surface, and activating T cells or NKT cells; therefore, dendritic cells are preferably used in the present invention. In the present invention, the antigen-presenting cells are preferably CD1 (for example, CD1d) expressing cells in order to secure the presentation of an NKT cell ligand to NKT cells.

Useful antigen-presenting cells are those derived from an optionally chosen mammal. Mammals include humans and non-human mammals. Examples of non-human mammals include rodents such as mice, rats, hamsters, and guinea pigs, laboratory animals such as rabbits, domestic animals such as pigs, bovines, goat, horses, and sheep, companion animals such as dogs and cats, primates such as monkeys, orangutans, and chimpanzees.

Although the genotype of the antigen-presenting cells contained in the agent of the present invention is not particularly limited, it is usually syngenic, allogenic or xenogenic relative to the subject to receive the agent of the present invention, preferably syngenic or allogenic. To avoid graft rejections, the antigen-presenting cells used are preferably syngenic relative to the subject to receive the agent of the present invention, more preferably derived from the subject to receive the agent of the present invention (i.e., autologous dendritic cells).

Antigen-presenting cells can be isolated from tissues (for example, lymph nodes, spleen, peripheral blood and the like) of the mammals mentioned above by a method known per se. For example, dendritic cells can be isolated using an antibody against a cell surface marker expressed specifically on antigen-presenting cells, by means of a cell sorter, panning, the antibody magnetic beads method and the like. When dendritic cells are isolated as antigen-presenting cells, CD11c, MHC Class I, MHC Class I-like molecules (CD1 and the like), MHC Class 11, CD8a, CD85k, CD86, FDL-M1, DEC-205 and the like can be used as cell surface markers expressed specifically on dendritic cells.

Antigen-presenting cells can also be produced by culturing bone marrow cells, mononuclear cells and the like of the mammals mentioned above under appropriate antigen-presenting cell differentiation conditions. For example, bone marrow cells, when cultured in the presence of GM-CSF (and IL-4 in some cases) for about 6 days, differentiate into dendritic cells (bone marrow-derived dendritic cells: BMDC) (Nature, 408, p. 740-745, 2000). By culturing mononuclear cells (particularly monocytes, macrophages and the like) in peripheral blood in the presence of GM-CSF (and IL-2 and/or IL-4 in some cases), dendritic cells can be obtained (References: Motohasi S, Kobayashi S, Ito T, Magara K K, Mikuni O, Kamada N, Iizasa T, Nakayama T, Fujisawa T, Taniguchi M, Preserved IFN-alpha production of circulating Valpha24 NKT cells in primary lung cancer patients, Int J Cancer, 2002, Nov. 10; 102(2): 159-165. Erratum in Int J Cancer. 2003, May 10; 104(6): 799).

“Pulse of antigen-presenting cells with an NKT cell ligand” refers to placing an NKT cell ligand on the antigen-presenting cell surface in a way that allows the ligand to be presented to NKT cells. More specifically, the same means presenting an NKT cell ligand onto a CD1 molecule expressed on the antigen-presenting cell surface. Pulse of antigen-presenting cells with an NKT cell ligand can be achieved by bringing the NKT cell ligand into contact with the antigen-presenting cells. For example, antigen-presenting cells are cultured in a physiological culture medium containing an NKT cell ligand. In this case, the concentration of the NKT cell ligand in the culture medium can be set as appropriate according to the kind of the NKT cell ligand, and is, for example, 1 to 10000 ng/ml, preferably 10 to 1000 ng/ml. Examples of culture mediums include basal media (minimum essential medium (MEM), Dulbecco's modified Eagle medium (DMEM), RPMI1640 medium, 199 medium) and the like, optionally containing appropriate additives (serum, albumin, buffers, amino acids and the like). The pH of the culture medium is usually about 6 to 8, cultivation temperature is usually about 30 to 40° C., and cultivation period is usually 4 to 14 days, preferably 6 to 14 days. Furthermore, after cultivation, by washing the antigen-presenting cells with a culture medium or physiological aqueous solution free of an NKT cell ligand to remove the free NKT cell ligand, antigen-presenting cells pulsed with the NKT cell ligand are isolated.

The agent of the present invention can contain antigen-presenting cells pulsed with an NKT cell ligand as the only active ingredient, or as a mixture with another optionally chosen active ingredient for treatment. The agent of the present invention can be produced by blending an effective amount of active ingredient with one or more kinds of pharmacologically acceptable carrier, by an optionally chosen method well known in the technical field of pharmaceutical making.

The agent of the present invention is usually provided in dosage forms such as injections and drip infusions. The agent of the present invention is preferably a suspension of antigen-presenting cells pulsed with an NKT cell ligand in a sterile aqueous carrier that is isotonic to the recipient's body fluid (blood and the like). The aqueous carrier is exemplified by physiological saline, PBS and the like. These aqueous carriers can further be supplemented with solubilizers, buffering agents, isotonizing agents, soothing agents, preservatives, stabilizers and the like as required.

The concentration of the antigen-presenting cells pulsed with an NKT cell ligand contained in the agent of the present invention usually falls in the range of, but is not limited to, about 1×10⁵ to 1×10¹⁰ cells/ml, preferably about 2×10⁵ to 1×10⁹ cells/ml. If the cell density is too low, a long time is taken for administration so that the burden on the patient increases; if the cell density is too high, the cells are likely to aggregate with each other.

The agent of the present invention is safe, and can be administered to an optionally chosen mammal. Mammals include the mammals mentioned above. The mammal is preferably a human.

The agent of the present invention is characterized by being submucosally administrated in the upper airway. The upper airway mucosa refers to the mucosa present on the surface of the upper airway from the nasal cavity to the trachea (nasal cavity, pharynx, tonsil, larynx, trachea and the like). Because immunocompetent cells and blood vessels are abundantly present in the nasal cavity mucosa, the agent of the present invention is preferably administered submucosally in the nasal cavity. The nasal cavity mucosa consists of the superior, middle, and inferior nasal concha mucosae, the superior, middle, and inferior nasal meatus mucosae, the nasal septal mucosa and the like; because of the abundance of immunocompetent cells and the ease of administration, the agent of the present invention is more preferably administered submucosally in the inferior nasal concha, still more preferably submucosally in the anterior portion of the inferior nasal concha mucosa. “Submucosal administration” refers to injecting an active ingredient into the lamina propria under mucosal epithelium.

The dosage of the agent of the present invention varies depending on dosage form, the patient's age and body weight, kind of disease, seriousness of disease, kind of NKT cell ligand and the like; usually, the agent of the present invention is administered at doses of usually 1×10⁶ to 1×10⁹ cells/m², preferably 1×10⁷ to 1×10⁹ cells/m², per time of administration, based on the number of antigen-presenting cells pulsed with an NKT cell ligand. However, these dosages vary depending on the various conditions described above.

By using the agent of the present invention, it is possible to induce NKT cells selectively in cervical lymph nodes. This selectivity is strict; NKT cells are induced selectively in cervical lymph nodes on the same side (ipsilateral) as the site of the upper airway mucosa where dendritic cells are administered. For example, when antigen-presenting cells pulsed with an NKT cell ligand are administered submucosally in the nasal cavity on the right side, NKT cells are induced selectively in cervical lymph nodes on the right side. Ligand-activated NKT cells have been reported to have a unique action mechanism to promptly produce large amounts of interferon γ and IL-4, to exhibit potent cytotoxic activity via perforin/granzyme B, and to subsequently induce a variety of immune reactions, resulting in a potent antitumor action [Morita M, Motoki K, Akimoto K, Natori T, Sakai T, Sawa E, Yamaji K, Koezuka Y, Kobayashi E, Fukushima H, Structure-activity relationship of alpha-galactosylceramides against B16-bearing mice. J Med Chem 1995 Jun. 9; 38 (12): 2176-87, Nakagawa R, Motoki K, Ueno H, Iijima R, Nakamura H, Kobayashi E, Shimosaka A, Koezuka Y, Treatment of hepatic metastasis of the colon26 adenocarcinoma with an alpha-galactosylceramide, KRN7000. Cancer Res 1998 Mar. 15; 58(6): 1202-7, Kawano T, Cui J, Koezuka Y, Toura I, Kaneko Y, Sato H, Kondo E, Harada M, Koseki H, Nakayama T, Tanaka Y, Taniguchi M, Natural killer-like nonspecific tumor cell lysis mediated by specific ligand-activated Valpha14 NKT cells. Proc Natl Acad Sci USA 1998 May 12; 95(10): 5690-3]. Therefore, by using the agent of the present invention, it is possible to induce immune responses mediated by NKT cells selectively in cervical lymph nodes; therefore, the agent of the present invention can be useful in the prophylaxis and treatment for malignant tumors in the head and neck region (nasal/paranasal sinus cancer, pharyngeal cancer, oral cancer, laryngeal cancer, thyroidal cancer, salivary gland cancer and the like), allergic diseases in the upper airway (nasal allergy and the like) and the like.

With the use of the agent of the present invention, it is possible to stimulate NKT cells and induce proliferation of NKT cells and production of cytokines (interferon γ, IL-4 and the like) extremely efficiently with a small number of NKT cell ligand-pulsed antigen-presenting cells. Particularly, by using the agent of the present invention, not only NKT cells in cervical lymph nodes, but also NKT cells in peripheral blood, can be stimulated extremely efficiently. Therefore, with the use of the agent of the present invention, it is possible to stimulate immune reactions extremely efficiently, and to prevent and treat diseases such as tumors and allergies, with a small number of NKT cell ligand-pulsed antigen-presenting cells.

The present invention is hereinafter described in more detail by means of the following examples, to which, however, the present invention is never limited.

### EXAMPLES

### Example 1

### Subjects

Patients with head and neck cancers who met the following criteria were selected.

### Selection Criteria;

1. Patients with advanced head and neck cancer (stage III, IV)
2. Age: 20 to 80 years
3. Performance status: 0 to 2
4. Patients meeting the following laboratory test value criteria (measurements taken within 4 weeks before registration)
WBC count ≧3,000/mL, platelet count ≧75,000/mL, serum creatine ≦1.5 mg/dL, total bilirubin ≦1.5 mg/dL, AST (GOT), ALT (GPT) ≦2.5× upper limit of criterion values
5. Written consent obtained from the patient or a proxy consenter
6. Patients having NKT cells in peripheral blood (not less than 10 cells/peripheral blood 1 ml)

### Exclusion Criteria;

1. Patients who have undergone chemotherapy or radiotherapy within 4 weeks before enrollment in the clinical study
2. Patients thought to have a prognosis of less than 6 months
3. Patients with active infectious disease
4. Patients with hepatitis or a past history thereof
5. Patients who are positive for HBs antigen, HCV antibody, HIV antibody or HTLV-1 antibody
6. Patients with concurrent double cancers
7. Patients with serious heart disease (NYHA Class III or higher)
8. Patients on any corticosteroid as a concomitant drug
9. Women who are pregnant or may become pregnant. Lactating women.
10. Patients with a past history of albumin hypersensitivity
11. Patients with autoimmune disease
12. Patients judged by the attending physician to be inappropriate for participation in the present clinical study because of a medical, psychological or any other factor

### (Methods)

### Preparation of α-GalCer-Pulsed Dendritic Cells

Peripheral blood (about 100 ml) was collected from each patient with head and neck cancer who met the above-described criteria. Furthermore, mononuclear cells were separated by density gradient centrifugation. Mononuclear cells in an amount sufficient to the dosage (the remaining was stored under freezing) were cultured in an AIM-V medium (Invitrogen Corp.) containing 800 U/ml GM-CSF (GeneTech Co., Ltd), 100 U/ml IL-2 (Immunase, Shionogi) and 5% autologous serum for 7 to 14 days. On the day before administration, 100 ng/ml α-GalCer (KRN7000; Kirin Brewery) was added, and the cells were cultured for 1 day to obtain α-GalCer-pulsed dendritic cells (DC). After washing, the cells were suspended in physiological saline supplemented with 2.5% albumin, and administered submucosally in the nasal cavity mucosa of the same patient.

### Route and Dose of Administration

The α-GalCer-pulsed dendritic cells were suspended in physiological saline (about 0.2 ml) supplemented with 2.5% albumin, and infused submucosally in the base of the inferior nasal concha of the patient. The dosage of the dendritic cells was 1×10⁸ cells/m².

On day 7 and day 14 in the 5-week study period, the α-GalCer-pulsed dendritic cells were administered submucosally in the nasal cavity.

### (Items for Evaluation)

### Evaluation of NKT Cell Counts

Blood was drawn weekly over 5 weeks before and after administration, and changes in the number of peripheral blood NKT cells were evaluated. The evaluation was performed by flowcytometry using the antibodies shown below. CD3⁺Vα24⁺Vβ11⁺ cells were defined as the NKT cells. The number of NKT cells per ml of peripheral blood was measured, and compared over time. CD3⁻CD56⁺ cells were defined as the NK cells, and the number of NK cells for control was measured over time.

Anti-human Vα24 mouse monoclonal antibody (C15; Immunotech)

Anti-human Vβ11 mouse monoclonal antibody (C21; Immunotech)

Anti-human CD3 mouse monoclonal antibody (UCTH1; PharMingen)

Anti-human CD56 mouse monoclonal antibody (Bectondickinson)

### Functional Evaluation of NKT Cells

Blood was drawn weekly over 5 weeks before and after administration, and peripheral blood mononuclear cells were separated and stored under freezing. At week 6, the cells were thawed, and the frequency of γ interferon-producing cells was measured with α-galactosylceramide by ELISPOT. ELISPOT assay was performed using a kit (manufactured by MABTECH) and a nitrocellulose membrane (Millititer; Millipore Corp.) as directed in the manufacturers' instruction manuals. The cells were stimulated by cultivation in a serum-free AIM-V medium containing 100 ng/ml α-GalCer for 18 hours. Color development was performed using the BCIP/NBT system (Bio-Rad). Spots were counted subjectively by computer image analysis.

### (Results)

Patient 1: 54-Year-Old Man. Recurrent Case of Middle Pharyngeal Cancer (T4N2cM1).

### Profile of Dendritic Cells

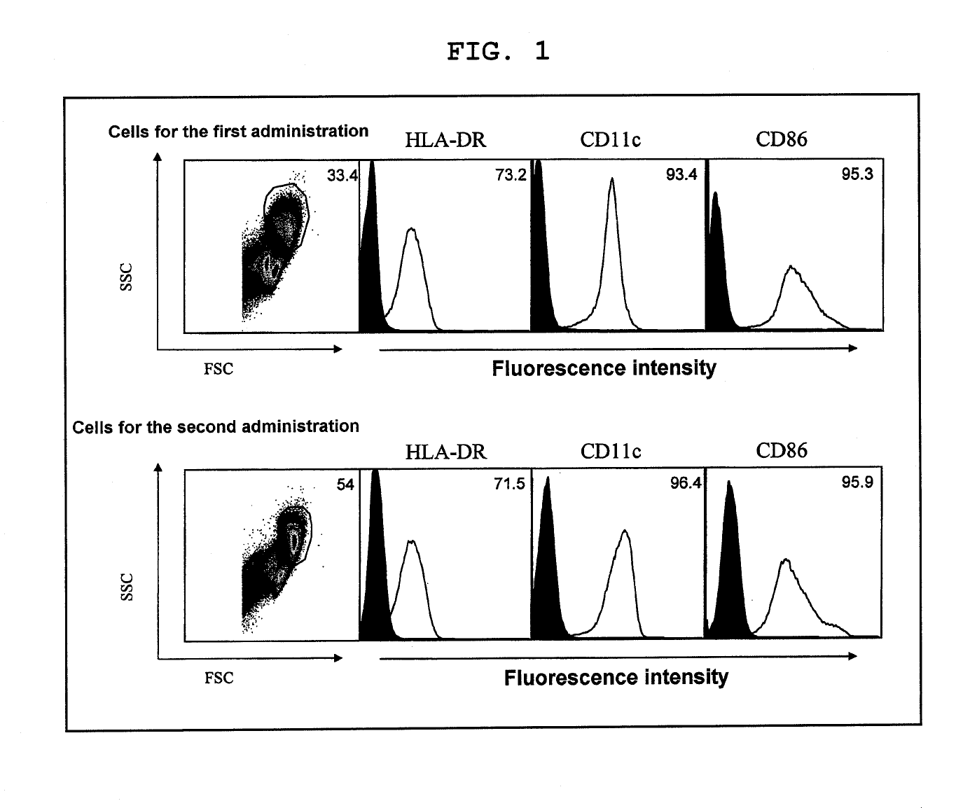

To obtain the profile of the dendritic cells administered, the expression of HLA-DR, CD11c, and CD86 on the cell surface was analyzed by flowcytometry; high expression of each surface antigen was confirmed (FIG. 1).

### Responses of Peripheral Blood NKT Cells

### 1) Quantitative Changes

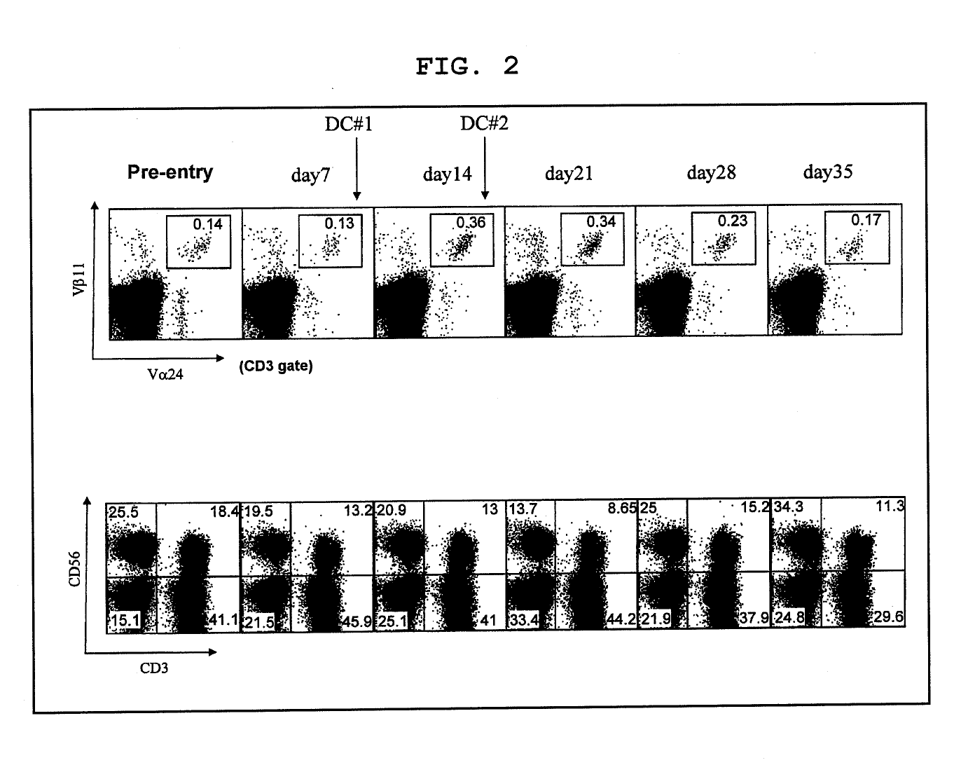

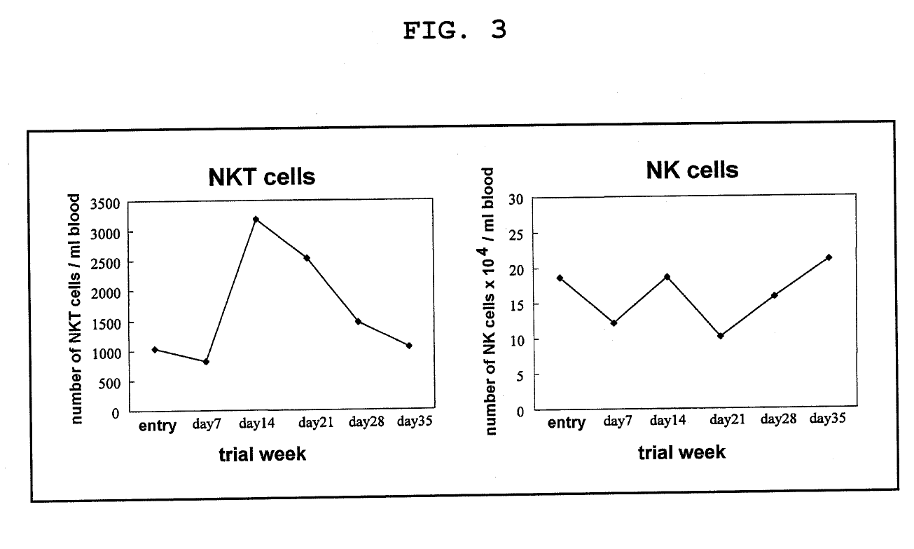

FIG. 2 shows NKT cells (upper panel) and NK cells (lower panel) in peripheral blood obtained by flowcytometry. Also measured were changes in the numbers of NKT cells and NK cells per ml (FIG. 3). By a single-dose submucosal administration of α-GalCer-pulsed dendritic cells in the nasal cavity, the number of peripheral blood NKT cells increased. Meanwhile, the number of peripheral blood NK cells did not change significantly with the administration of α-GalCer-pulsed dendritic cells.

### 2) Functional Changes

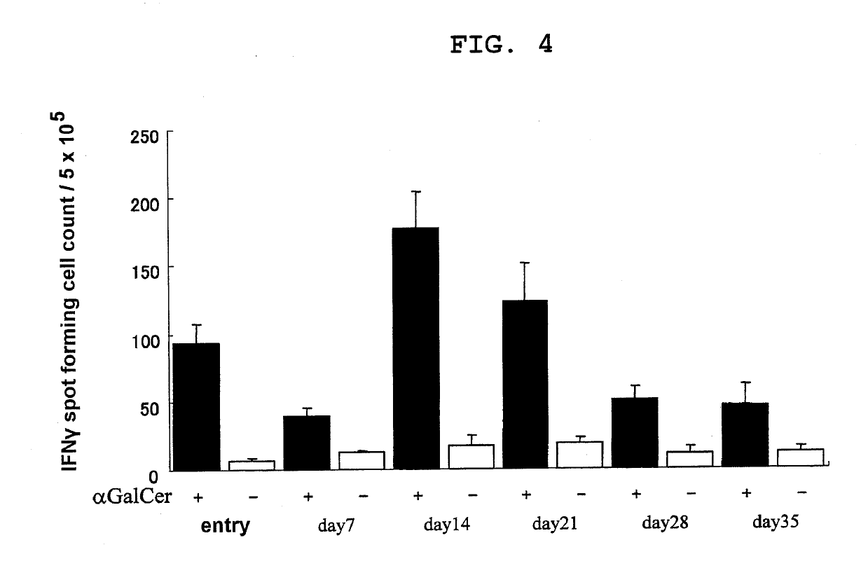

FIG. 4 shows changes in the number of cells that produced γ interferon in response to α-GalCer stimulation, contained in a peripheral blood mononuclear cell fraction obtained by ELISPOT. Proportionally in the number of peripheral blood NKT cells, the number of γ interferon-producing cells increased in to response to the administration of α-GalCer-pulsed dendritic cells.

Patient 2: 48-Year-Old Woman. Recurrent Case of Left Maxillary Cancer (T3N0M0).

### Profile of Dendritic Cells

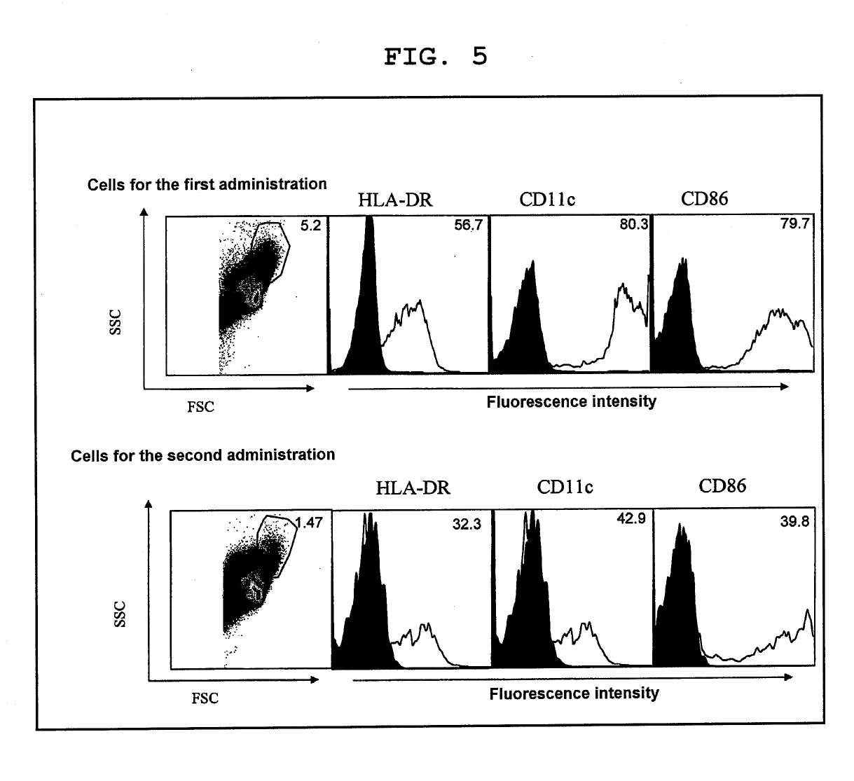

To obtain the profile of the dendritic cells administered, the expression of HLA-DR, CD11c, and CD86 on the cell surface was analyzed by flowcytometry; the expression of each surface antigen was confirmed (FIG. 5).

### Responses of Peripheral Blood NKT Cells

### 1) Quantitative Changes

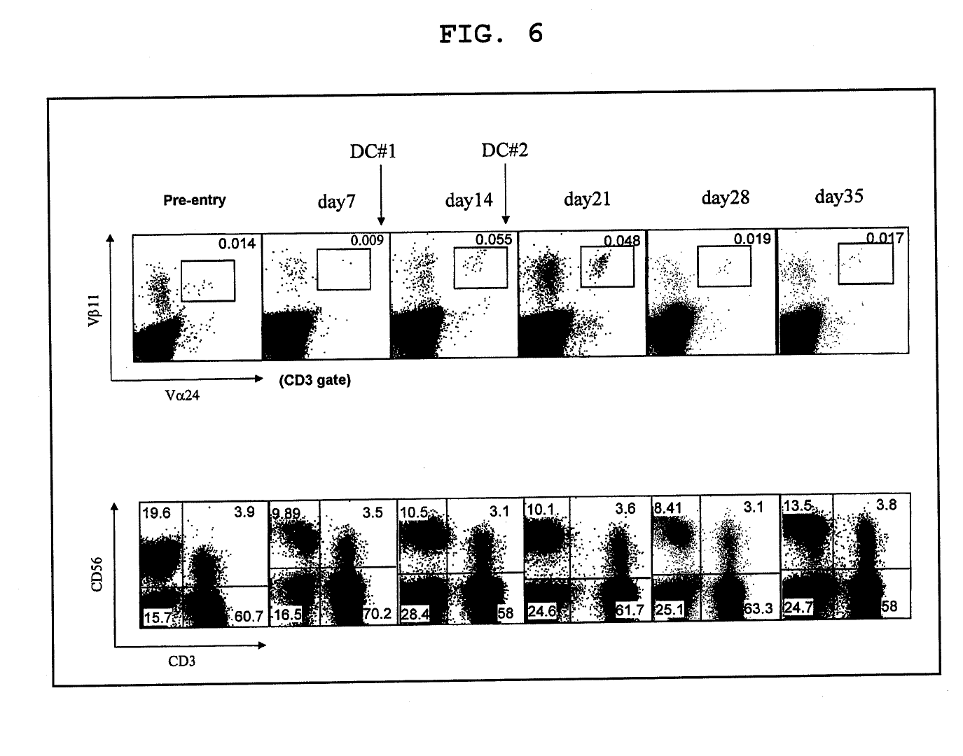

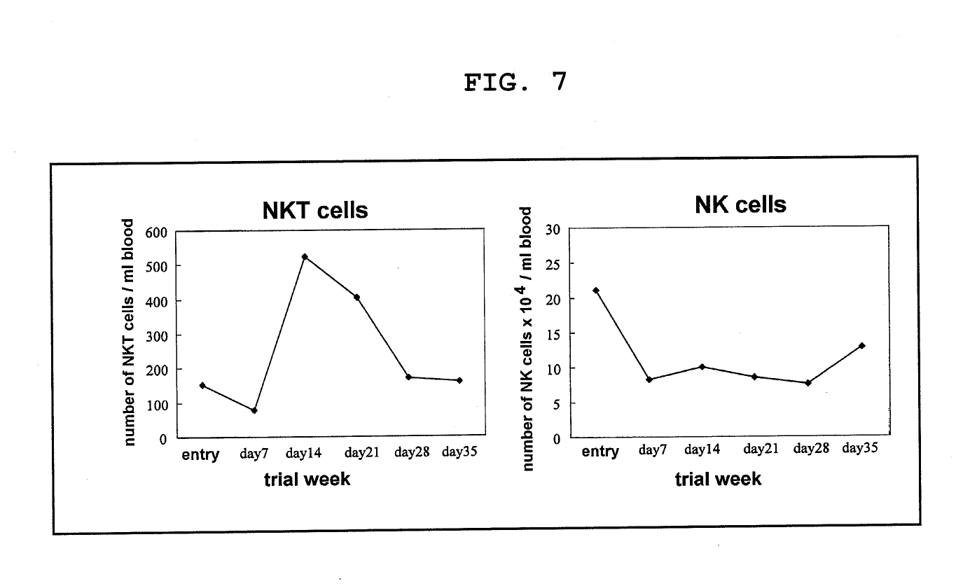

FIG. 6 shows NKT cells (upper panel) and NK cells (lower panel) in peripheral blood obtained by flowcytometry. Also measured were changes in the numbers of NKT cells and NK cells per ml (FIG. 7). By a single-dose submucosal administration of α-GalCer-pulsed dendritic cells in the nasal cavity, the number of peripheral blood NKT cells increased. Meanwhile, the number of peripheral blood NK cells did not change significantly with the administration of α-GalCer-pulsed dendritic cells.

### 2) Functional Changes

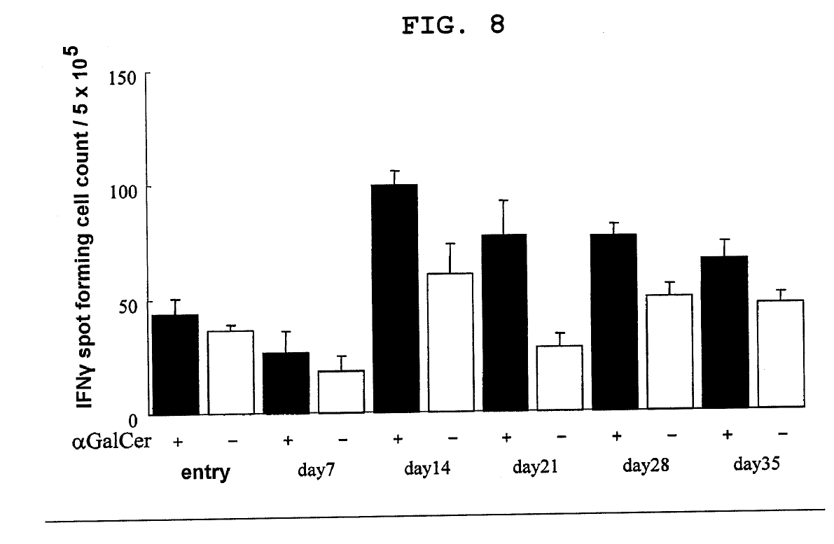

FIG. 8 shows changes in the number of cells that produced γ interferon in response to α-GalCer stimulation, contained in a peripheral blood mononuclear cell fraction obtained by ELISPOT. Proportionally in the number of peripheral blood NKT cells, the number of γ interferon-producing cells increased in response to the administration of α-GalCer-pulsed dendritic cells.

To date, mainly in recurrent cases of lung cancer, intravenous administration of α-galactosylceramide-pulsed dendritic cells has been investigated [Ishikawa A, Motohashi S, Ishikawa E, Fuchida H, Higashino K, Otsuji M, Iizasa T, Nakayama T, Taniguchi M, Fujisawa T, A phase I study of alpha-galactosylceramide (KRN7000)-pulsed dendritic cells in patients with advanced and recurrent non-small cell lung cancer. Clin Cancer Res. 2005 Mar. 1; 11(5): 1910-7]. According to the investigation, a phase 1 study was performed with escalation of the number of transferred cells from 5×10⁷/m² for level 1 to 2.5×10⁸/m² for level 2 and 1×10⁹/m² for level 3. As a result, of the 11 patients who participated in the study, one receiving level 3 cells had an increased number of peripheral blood NKT cells. However, with α-galactosylceramide-pulsed dendritic cells of level 1 and level 2 numbers, no immune responses for increased NKT cells in peripheral blood were obtained.

In contrast, when α-galactosylceramide-pulsed dendritic cells were administered submucosally in the upper airway as shown in the Examples, an increased number of NKT cells in peripheral blood was observed at a small dose of 1×10⁸/m². Furthermore, not only quantitatively, but also functionally, the cytokine (interferon γ) production response of NKT cells to α-GalCer was enhanced.

From these results, it was shown that by administering NKT cell ligand-pulsed antigen-presenting cells submucosally in the upper airway, peripheral NKT cells could be stimulated extremely efficiently with a small number of NKT cell ligand-pulsed antigen-presenting cells.

### Example 2

α-GalCer-pulsed dendritic cells prepared in the same manner as Example 1 were suspended in physiological saline (about 0.2 ml) supplemented with 2.5% albumin, and infused submucosally in the base of the inferior nasal concha in the left nasal cavity of each patient with head and neck cancer. The dosage of the dendritic cells was 1×10⁸ cells/m². Two days after administration, lymphocytes were collected from the cervical lymph nodes on both sides by biopsy, and examined for the presence or absence of NKT cells in the collected lymphocytes by flowcytometry in the same manner as Example 1. CD3⁺Vα24⁻Vβ11⁺ cells were defined as the NKT cells.

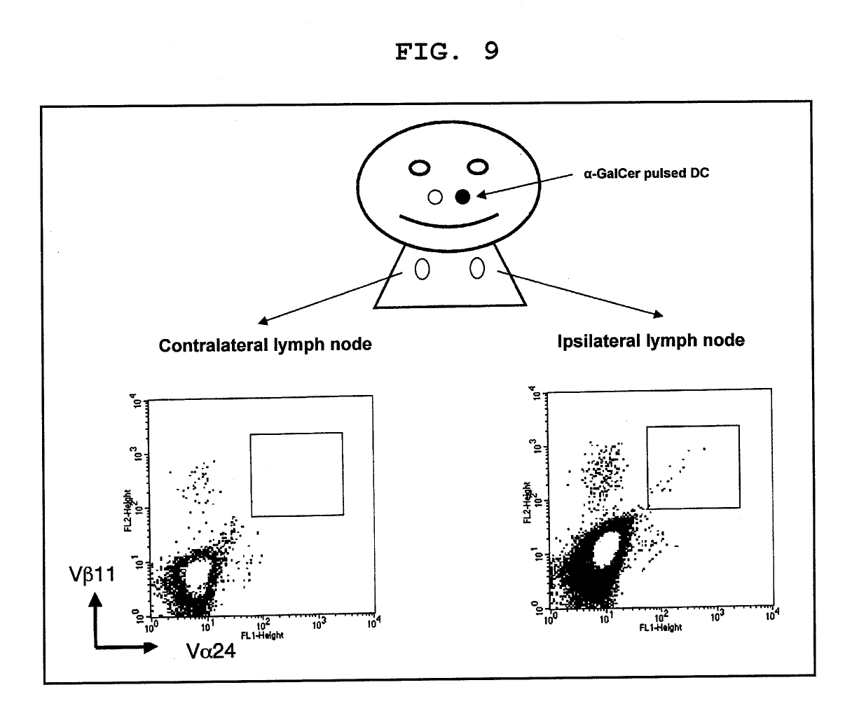

As a result, the presence of NKT cells was observed in the cervical lymph nodes on the same side as the site of administration of α-GalCer-pulsed dendritic cells, but the presence of NKT cells was not observed in the contralateral cervical lymph nodes (FIG. 9).

From these results, it was shown that by submucosal administration of α-GalCer-pulsed dendritic cells in the upper airway, NKT cells were induced selectively in cervical lymph nodes.

### Reference Example 1

In the same manner as Examples 1 and 2, lymphocytes were collected from peripheral blood and non-metastatic cervical lymph nodes of each patient with head and neck cancer, and examined by flowcytometry for the presence or absence of NKT cells in the collected lymphocytes. CD3⁺Vα24⁺Vβ11⁺ cells were defined as the NKT cells.

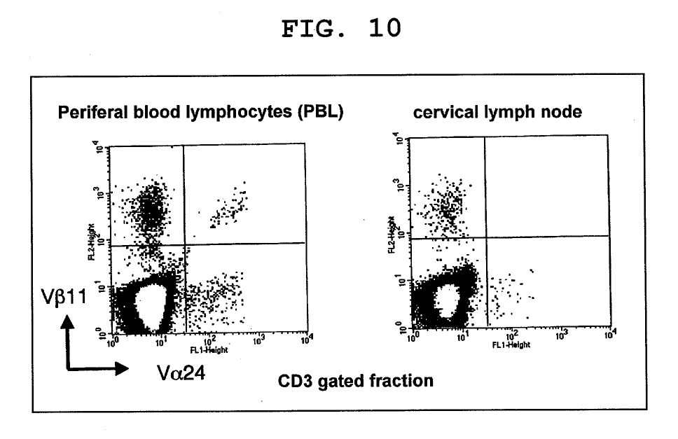

As a result, the presence of NKT cells was observed in the lymphocytes in peripheral blood, whereas no NKT cells were detected in the non-metastatic lymph nodes (FIG. 10).

### INDUSTRIAL APPLICABILITY

With the use of the agent of the present invention, it is possible to stimulate NKT cells, stimulate immune reactions, and treat diseases such as cancer extremely efficiently with a small number of NKT cell ligand-pulsed antigen-presenting cells. This allows a significant reduction in the consumption of reagents used to prepare antigen-presenting cells, thus cutting the costs of the treatment as a whole. Additionally, because the amount of mononuclear cells collected from the patient to prepare antigen-presenting cells can be reduced, and also because the time taken to administer antigen-presenting cells is shortened, the burden on the patient is lessened. Furthermore, because the amount of NKT cell ligand required for the treatment also decreases significantly, safety in the treatment improves further.

Furthermore, with the use of the agent of the present invention, it is possible to induce NKT cells selectively in cervical lymph nodes and activate antitumor immunity via NKT cells in the cervical lymph nodes.

This application is based on a patent application No. 2005-294124 filed in Japan (filing date: Oct. 6, 2005), the contents of which are incorporated in full herein by this reference.

1. An NKT cell stimulating agent containing antigen-presenting cells pulsed with an NKT cell ligand, to be administered submucosally in an upper airway.

2. The agent of claim 1, wherein the NKT cell ligand is α-galactosylceramide.

3. The agent of claim 1, wherein the upper airway mucosa is a nasal cavity mucosa.

4. An inducing agent of NKT cells in cervical lymph nodes containing antigen-presenting cells pulsed with an NKT cell ligand, to be administered submucosally in an upper airway.

5. The agent of claim 4, wherein the NKT cell ligand is α-galactosylceramide.

6. The agent of claim 4, wherein the upper airway mucosa is the nasal cavity mucosa.

7. An inducing agent of interferon γ production containing antigen-presenting cells pulsed with an NKT cell ligand, to be administered submucosally in an upper airway.

8. The agent of claim 7, wherein the NKT cell ligand is α-galactosylceramide.

9. The agent of claim 7, wherein the upper airway mucosa is a nasal cavity mucosa.

10. An immunostimulating agent containing antigen-presenting cells pulsed with an NKT cell ligand, to be administered submucosally in an upper airway.

11. The agent of claim 10, wherein the NKT cell ligand is α-galactosylceramide.

12. The agent of claim 10, wherein the upper airway mucosa is a nasal cavity mucosa.

13. A method of stimulating NKT cells, comprising administering antigen-presenting cells pulsed with an NKT cell ligand submucosally in an upper airway.

14. A method of inducing NKT cells in cervical lymph nodes, comprising administering antigen-presenting cells pulsed with an NKT cell ligand submucosally in an upper airway.

15. A method of inducing interferon γ production, comprising administering antigen-presenting cells pulsed with an NKT cell ligand submucosally in an upper airway.

16. A method of stimulating immune reactions, comprising administering antigen-presenting cells pulsed with an NKT cell ligand submucosally in an upper airway.

17. A use of antigen-presenting cells pulsed with an NKT cell ligand, for producing an NKT cell stimulating agent to be administered submucosally in an upper airway.

18. A use of antigen-presenting cells pulsed with an NKT cell ligand, for producing an inducer of NKT cells in cervical lymph nodes to be administered submucosally in an upper airway.

19. A use of antigen-presenting cells pulsed with an NKT cell ligand, for producing an inducing agent of interferon γ production to be administered submucosally in an upper airway.

20. A use of antigen-presenting cells pulsed with an NKT cell ligand, for producing an immunostimulant to be administered submucosally in an upper airway.
